(19) United States
(12) Patent Application Publication
(10) Pub. No.: US 2022/0354123 A1
(43) Pub. Date: Nov. 10, 2022

(54) MELANOIDIN COMPOSITIONS FOR PROTECTING CROPS FROM NONFUNGAL PESTS

(71) Applicant: THE STATE OF ISRAEL, MINISTRY OF AGRICULTURE & RURAL DEVELOPMENT, AGRICULTURAL RESEARCH ORGANIZATION, Rishon Lezion (IL)

(72) Inventors: Yigal ELAD, Ganei Tiqva (IL); Ellen Ruth GRABER, Rehovot (IL)

(21) Appl. No.: 17/622,576

(22) PCT Filed: Jun. 25, 2020
(86) PCT No.: PCT/IL2020/050709

§ 371 Date: Dec. 23, 2021

(30) Foreign Application Priority Data
Jun. 25, 2019 (IL) 267627

(51) Int. Cl. A01N 61/00; A01P 1/00; A01P 7/04; A01P 7/00

(52) U.S. Cl. CPC A01N 61/00; A01P 1/00; A01P 7/04; A01P 7/00

(57) ABSTRACT

Use of brown melanoidins in compositions and methods to protect plants against a wide variety of non-fungal pests is disclosed. In some exemplary embodiments of the invention, the plants are nightshades, for example tomato. In some exemplary embodiments of the invention, the plants are cucurbits, for example cucumber. Alternatively or additionally, in some embodiments the pests are selected from the group consisting of viruses, bacteria, oomycetes, and arthropods.

### FIELD OF THE INVENTION

The invention is in the field of crop protection.

### BACKGROUND OF THE INVENTION

Nightshades and cucurbits constitute economically important families of plants. Nightshades include, for example, potato, tomato, pepper, eggplant and tobacco. Cucurbits include, for example, cucumbers, squash, pumpkin and watermelon. Potatoes have become a staple food, being the world's fourth-largest food crop. Tomatoes are the largest vegetable crop, grown around the world, including in greenhouses throughout the year, being used fresh, in salads, dishes, ketchup, etc. Cucumbers are in the top five produced vegetables worldwide. Nightshades and cucurbits are an important part of human diet, providing basic energy nutrients, additives, vitamins, and minerals. Widely spread pathogens reduce the quality and quantity of the harvested products. The problem is that the pests may belong to diverse kingdoms such as archaea pests, bacteria, oomycetes, fungi, and animals, as well as pathogens that do not belong on the tree of life, because they do not have independent means of reproduction, such as viruses. This makes plant protection very complex. Ever-increasing environmental concerns make plant protection still more difficult, because the use of broadly acting toxins is continually restricted, as well as the concentrations thereof.

### SUMMARY OF THE INVENTION

One aspect of some embodiments of the invention relates to a composition comprising melanoidin(s). According to various exemplary embodiments of the invention the composition includes stimulants and/or pesticides and/or adjuvants for protecting crops from nonfungal pests. In some exemplary embodiments of the invention, the melanoidin is obtained by reacting an amino acid source and a reducing sugar source in solid phase at a temperature of from about 120° C. to 180° C., wherein the extent of melanoidinization can be followed spectrophotmetrically by the development of the brown color, as usually determined for melanoidins at 420 nm. In other exemplary embodiments of the invention, the reaction is performed in a liquid phase, such as in solution or in suspension. The term melanoidinization refers to the Maillard reaction in which a carbonyl group of a sugar reacts with an amino group of an amino acid at an elevated temperature while forming brown oligomers and polymers which can be detected spectrophotometrically. Over the time course of a reaction, the absorbance at 420 nm reaches a plateau for an aqueous solution made up from the heated amino acid-reducing sugar mixture to a given concentration, assuming that the said concentration gives a linear response in UV-Vis following the limitations of the Beer-Lambert law. In some embodiments of the composition, the melanoidin is obtained by reacting an amino acid source and a reducing sugar source at a molar ratio of between 0.5 and 2.0, in solid phase at a temperature of from 120 to 180° C., such as from 130 to 170° C. or from 135 to 165° C., wherein the extent of melanoidinization reaches an endpoint, which may be defined spectrophotometrically. In some embodiments, the extent is between 0.75 and 1 or between 0.9 and 1 or between 0.95 and 1, for example about 1. Optionally, the melanoidin is prepared by reacting an amino acid source, a reducing sugar source, reaction products of the sources when heated in an approximately equimolar ratio at a temperature of from 120 to 180° C., and an amount of solvent, wherein the extent of the reaction at said temperature can be determined spectrophotometrically. Said amino acid source may comprise one or more amino acids, one or more peptides with a free amino group, or a protein hydrolysate. Said reducing sugar source may comprise one or more monosaccharides, one or more disaccharides, one or more oligosaccharides, or mixtures thereof.

In another aspect of some embodiments of the invention, the composition comprising melanoidin(s) protects nightshade and/or cucurbit crops from nonfungal pests. One example of a nightshade crop is tomato. One example of a curcubit crop is cucumber. According to various exemplary embodiments of the invention the pests include viruses and/or bacteria and/or oomycetes and/or arthropods. In some exemplary embodiments of the invention, the composition protects against at least two different pests selected from viruses, bacteria, oomycetes, and arthropods.

In some embodiments the composition is formulated as a powder which can be safely stored for prolonged periods without losing efficiency, and easily transferred to the user. According to these embodiments the powder is diluted with a solvent to provide a working composition.

Alternatively or additionally, the composition is formulated as a suspension or solution to be stored for later use and/or for further dilution and/or for applying onto the plants in need of protection. In some embodiments the solution or suspension applied onto the plants contains said melanoidins at an amount of from 0.001 to 4 wt %, for example from 0.002 to 2 wt %, for example from 0.01 to 1 wt %.

In another aspect of the invention, the melanoidin composition also contains additional agriculturally active components, such as pesticides and/or stimulants and/or adjuvants and/or auxiliary additives. Nonlimiting examples of additional active components include pyrimidine-based agents, phenylpyrrole-based agents, alkyl phenol ethylene oxide condensate, pyrimethanil, cyprodinil, and fludioxonil.

Another aspect of the invention relates to a method for controlling damage caused in a plant by nonfungal pests and/or for protecting crops from nonfungal pests, comprising i) providing an amino acid source comprising one or more amino acids, one or more peptides, or a protein hydrolysate; ii) providing a reducing sugar source selected from one or more monosaccharides, one or more disaccharides, one or more oligosaccharides, or mixtures thereof; iii) combining said amino acid source with said reducing sugar source at a molar ratio of between 0.5 and 2.0 to form a homogeneous mixture, optionally with an amount of solvent, for example water; iv) heating the combined materials at a temperature of from 100° C. to 180° C., for example 110-180 or about 120-180 or 110-170 or 120-170 or 120-160° C., for a time period sufficient to form the desired amount of the brown melanoidin product, by reacting said amino acid source with said sugar source, the reaction being called melanoidinization and being measured and followed-up spectrophotometrically after desired dilution in water; iv) diluting said melanoidins in solvents to a desired working concentration, and optionally admixing additional components selected from stimulants, pesticides, and adjuvants, thereby obtaining a melanoidin working composition; and v) applying to said plant an effective amount of said melanoidin working composition. Melanoidinization is performed, for example, at a molar ratio between said amino acid source and said sugar source of about 1 to 1. In one embodiment, the solvent is water and the amount of added water does not result in formation of separate phases. In other embodiments, the added solvent results in the formation of homogenizable suspension. Controlling damage or protecting crops in the method of the invention comprises preventing a damage caused by said pest, improving the resistance of said plant or crops to said pest, or reducing the symptoms of a disease caused by said pest or reducing the damages caused by said disease. The method of the invention protects the plants against pests selected from viruses, bacteria, oomycetes, and arthropods. In some embodiments plants or crops are protected against at least two different pests. In some embodiments the method of the invention employs the melanoidin composition as a liquid formulation, for example an aqueous solution, optionally at a concentration of from 0.002 to 2 wt %, preferably from 0.01 to 1 wt %, by spray, sprinkle, drench, irrigation, or fertigation.

Some exemplary embodiments of the invention provide a composition for reducing damages on nightshade and/or curcubit crops caused by nonfungal pests, the composition comprising melanoidins and optionally additional pesticides or adjuvants or stimulants.

It will be appreciated that the various aspects described above relate to solution of technical problems associated with soil and/or groundwater contamination resulting from use of chemical pesticides.

Alternatively or additionally, it will be appreciated that the various aspects described above relate to solution of technical problems related to protecting crops against a wide variety of non-fungal pests and/or diseases. In some embodiments the crops are members of the nightshade family and in some embodiments are members of the curcubit family.

In some exemplary embodiments of the invention there is provided a composition including a melanoidin. In some embodiments the composition includes at least one additional component selected from the group consisting of insecticides, acaricides and antiviral compounds. Alternatively or additionally, in some embodiments the melanoidin is obtained by reacting an amino acid source and a reducing sugar source in solid phase or in solution or in suspension at a temperature of from 110° C. to 180° C. Alternatively or additionally, in some embodiments the reaction proceeds at least 20%; at least 30%; at least 40%; at least 50%; at least 60%; at least 70%; at least 80%; at least 90%; at least 95% or substantially 100% to completion as evaluated spectrophotometrically. Alternatively or additionally, in some embodiments the amino acid source includes one or more amino acids, one or more peptides with a free amino group, or a protein hydrolysate. Alternatively or additionally, in some embodiments the reducing sugar source includes one or more monosaccharides, one or more disaccharides, one or more oligosaccharides, or mixtures thereof. Alternatively or additionally, in some embodiments the composition is provided as a powder. Alternatively or additionally, in some embodiments the composition is provided as a solution or a suspension in which the melanoidin constitutes from 0.002 to 2 wt %, to be used as a working composition. Alternatively or additionally, in some embodiments the melanoidin constitutes from 0.01 to 1 wt %. Alternatively or additionally, in some embodiments the additional component Is selected from pyrimidine-based agents, phenylpyrrole-based agents, alkyl phenol ethylene oxide condensate, pyrimethanil, cyprodinil, or fludioxonil. Alternatively or additionally, in some embodiments the composition is labeled for use in treatment of a nightshade or curcubit crop for a non-fungal pest. Alternatively or additionally, in some embodiments the nightshade crop is tomato. Alternatively or additionally, in some embodiments the curcubit crop is cucumber. Alternatively or additionally, in some embodiments the composition is for use in the treatment of a nightshade crop for a non-fungal pest. Alternatively or additionally, in some embodiments the non-fungal pest includes at least one member of the group consisting of viruses, bacteria, oomycetes, and arthropods. Alternatively or additionally, in some embodiments the pests comprise at least two different pests selected from the group.

In some exemplary embodiments of the invention there is provided a method including applying a formulation including a brown melanoidin product to crops infected by, or at risk for infection by, at least one member of the group consisting of viruses, bacteria, oomycetes, and arthropods. In some embodiments of the method, the brown melanoidin product is the result of reaction of an amino acid with a reducing sugar at a molar ratio of between 0.5 and 2.0 at a temperature of from 120° C. to 180° C., for a time period sufficient to achieve melanoidinization between 0.2 and 1 when measured spectrophotometrically. Alternatively or additionally, in some embodiments of the method, the formulation is applied as an aqueous solution with a brown melanoidin concentration of from 0.002 to 2 wt %. Alternatively or additionally, in some embodiments the brown melanoidin concentration is from 0.01 to 1 wt %. Alternatively or additionally, in some embodiments the applying is via an application route selected from the group consisting of spraying, sprinkling, drenching, irrigation, and fertigation.

In some exemplary embodiments of the invention there is provided a melanoidin composition for use in the treatment of a nightshade or curcubit crop to protect against a non-fungal pest. In some embodiments the non-fungal pest includes at least one member of the group consisting of viruses, bacteria, oomycetes, and arthropods. Alternatively or additionally, in some embodiments the nightshade crop is tomato. Alternatively or additionally, in some embodiments the non-fungal pest is Tomato brown rugose fruit virus (TBRFV). Alternatively or additionally, in some embodiments the non-fungal pest is Tomato yellow leaf curl virus (TYLCV). Alternatively or additionally, in some embodiments the non-fungal pest is the bacterium Pseudomonas syringae pv. (optionally Tomato). Alternatively or additionally, in some embodiments the non-fungal pest is the insect Tuta absoluta. Alternatively or additionally, in some embodiments the non-fungal pest is the insect Bemisia tabaci Gennadius. Alternatively or additionally, in some embodiments the non-fungal pest is the mite Tetranychus urticae. Alternatively or additionally, in some embodiments the non-fungal pest is the oomycete Phytophthora infestans. Alternatively or additionally, in some embodiments the curcubit crop is cucumber. Alternatively or additionally, in some embodiments the non-fungal pest is the oomycete Pseudoperonospora cubensis. Alternatively or additionally, in some embodiments the non-fungal pest is the oomycete Pythium aphanidermatum.

Unless otherwise defined, all technical and scientific terms used herein have the same meaning as commonly understood by one of ordinary skill in the art to which this invention belongs. Although suitable methods and materials are described below, methods and materials similar or equivalent to those described herein can be used in the practice of the present invention. In case of conflict, the patent specification, including definitions, will control. All materials, methods, and examples are illustrative only and are not intended to be limiting.

As used herein, the terms “comprising” and “including” or grammatical variants thereof are to be taken as specifying inclusion of the stated features, integers, actions or components without precluding the addition of one or more additional features, integers, actions, components or groups thereof. This term is broader than, and includes the terms “consisting of” and “consisting essentially of” as defined by the Manual of Patent Examination Procedure of the United States Patent and Trademark Office. Thus, any recitation that an embodiment “includes” or “comprises” a feature is a specific statement that sub embodiments “consist essentially of” and/or “consist of” the recited feature.

The phrase “consisting essentially of” or grammatical variants thereof when used herein are to be taken as specifying the stated features, integers, steps or components but do not preclude the addition of one or more additional features, integers, steps, components or groups thereof but only if the additional features, integers, steps, components or groups thereof do not materially alter the basic and novel characteristics of the claimed composition, device or method.

The phrase “adapted to” as used in this specification and the accompanying claims imposes additional structural limitations on a previously recited component.

The term “method” refers to manners, means, techniques and procedures for accomplishing a given task including, but not limited to, those manners, means, techniques and procedures either known to, or readily developed from known manners, means, techniques and procedures by practitioners of architecture and/or computer science.

Percentages (%) of chemicals typically are W/W (weight per weight) unless otherwise indicated.

### BRIEF DESCRIPTION OF THE DRAWINGS

In order to understand the invention and to see how it may be carried out in practice, embodiments will now be described, by way of non-limiting example only, with reference to the accompanying figures. In the figures, identical and similar structures, elements or parts thereof that appear in more than one figure are generally labeled with the same or similar references in the figures in which they appear. Dimensions of components and features shown in the figures are chosen primarily for convenience and clarity of presentation and are not necessarily to scale. The attached figures are:

FIGS. 1A and 1B are bar graphs showing the effect of a melanoidin composition according to one embodiment of the invention on a viral disease (Tomato brown rugose fruit virus, TBRFV) of tomato plants, FIG. 1A shows TBRFV severity, and FIG. 1B incidence of virus presence;

FIG. 2 is a bar graph showing the effect of a melanoidin composition according to one embodiment of the invention on a viral disease (Tomato yellow leaf curl virus, TYLCV) of tomato plants;

FIG. 3 is a bar graph showing the effect of a melanoidin composition according to one embodiment of the invention on a bacterial disease caused by Pseudomonas syringae pv. tomato of tomato plants;

FIG. 4 is a bar graph showing the effect of the melanoidin composition according to one embodiment of the invention on an arthropod-caused damage (insect Tuta absoluta) of tomato plants;

FIG. 5 is a bar graph showing the effect of the melanoidin composition according to one embodiment of the invention on an arthropod-caused damage (insect Bemisia tabaci) of tomato plants;

FIG. 6 is a bar graph showing the effect of the melanoidin composition according to one embodiment of the invention on an arthropod-caused damage (arachnid Tetranychus urticae) of tomato plants;

FIG. 7 is a bar graph showing the effect of a combined treatment according to one embodiment of the invention, comprising melanoidin composition and alkylphenol ethylene oxide condensate, in protecting tomatoes against arthropod-caused disease (Tetranychus urticae);

FIG. 8A is a histogram of absorbance units as a function wavelength (nm) for solutions obtained by heating a solid mixture of glucose and glutamic acid (called M11) in a molar ratio of 1:1 at 170° C. for different time intervals and mixing with water to a concentration of 250 mg/L; and

FIG. 8B (inset) is a histogram of absorbance units at 420 nm as a function of time for the same reaction as in FIG. 8A.

### DETAILED DESCRIPTION OF EMBODIMENTS

Embodiments of the invention relate to melanoidin compositions and methods to make and use them.

Specifically, some embodiments of the invention can be used to protect crops against damage caused by viruses and/or bacteria and/or oomycetes and/or arthropods.

The principles and operation of a compositions and/or methods according to exemplary embodiments of the invention may be better understood with reference to the drawings and accompanying descriptions.

Before explaining at least one embodiment of the invention in detail, it is to be understood that the invention is not limited in its application to the details set forth in the following description or exemplified by the Examples. The invention is capable of other embodiments or of being practiced or carried out in various ways. Also, it is to be understood that the phraseology and terminology employed herein is for the purpose of description and should not be regarded as limiting.

It has been surprisingly discovered that melanoidin compositions comprising melanoidins and/or amino acids and/or sugars in water efficiently protect nightshade crops, (e.g. tomatoes) and/or cucurbits (e.g. cucumbers), against damages caused by viruses and/or bacteria and/or oomycetes and/or arthropods.

Melanoidins are products of the Maillard reaction, which occurs in various food products containing sugars and amines during their heating, and which results in the brown color of baked food, beer, coffee, and many others. WO 2014/111932, which is fully incorporated herein by reference, describes the use of melanoidins for improving plant growth, drought tolerance, and resistance to a fungal pathogen. Due to the extremely unpredictable nature of biology, it was surprising to discover that melanoidins exhibit significant activity against a wide range of non-fungal pathogens and/or pests.

Various exemplary embodiments of the invention relate to employing a composition including melanoidin obtained by reacting an amino acid source and a reducing sugar source at a temperature of from 120 to 180° C., the extent of melanoidinization being determined spectrophotometrically, for protecting nightshade and curcubit crops from pathogens and/or pests not belonging to the fungi kingdom, wherein the amino acid source and the reducing sugar source are in a molar ratio of from 0.5 to 2.0. According to various exemplary embodiments of the invention the composition is formed by heating one or more amino acids, one or more peptides having a free amino group, a protein hydrolysate, or a mixture containing any one of them, with one or more reducing saccharides selected from one or more monosaccharides, one or more disaccharides, one or more oligosaccharides, or a mixture containing any of them. The heating time is selected to enable the formation of brown melanoidin products; for example, for temperatures of between 120° C. to 180° C., such as 130 or 140 or 150 or 160 or 170° C., the heating time may be between 2 minutes and 6 hours, for example between 5 minutes and 3 hours, while taking into consideration the heating equipment, and the type and amount of the raw material, so that the desired temperature may be achieved throughout the whole mixture volume and the extent of melanoidinization can be determined, for example, spectrophotometrically. The method and composition described herein are useful for improving plants' abilities to resist damages and diseases caused by nonfungal pests and pathogens. Without wishing to be limited by any particular theory, the inventors believe that melanoidins act as a biostimulant inducing or supporting the plant's natural defense pathways.

The composition comprises melanoidins and their source, and can be applied by spray, drench, irrigation, fertigation, or any other way of application.

Melanoidins for the use according to the invention can be prepared in various ways, including heating at normal or elevated pressure in various diluents or solvents or as dry solids. One method comprises heating a mixture containing at least one reducing sugar and an amino acid with a free amino group at a temperature of between 120 and 180° C. In one embodiment, the method comprises the steps of: (a) providing an amino acid with a free amino group; (b) providing a reducing sugar; (c) combining the two materials to form a homogeneous mixture; and (d) heating the combined materials at a temperature sufficient to create the desired amount of the brown product, measurable spectrophotometrically after dilution to a desired melanoidin concentration in a solvent (e.g. water). In some embodiments dilution is done in stages, for example by preparing a stock mixture at a high concentration then diluting the stock mixture to a lower concentration prior to use. In one preferred embodiment, (a) provided is a powder comprising an amino acid with a free amino group; (b) provided is a powder comprising a reducing sugar; (c) the two powders are combined to form a homogeneous solid mixture; and (d) the combined solid is heated at a temperature of from 120 to 180° C. for the time sufficient to create the desired amount of the brown product. In one embodiment said step (c) of combining the powders may comprise adding a solvent, such as water or an aqueous solution. In another embodiment, the materials in steps (c) and (d) form a liquid mixture. Said amino acid may be a free amino acid or amino acid incorporated into a peptide; said reducing sugar may be a monosaccharide, disaccharide, or an oligosaccharide. According to various exemplary embodiments the method employs, for example, an amino acid, a mixture of amino acids, or a mixture of peptides. In some embodiments the mixture of peptides is provided as a protein hydrolysate. According to various exemplary embodiments of the invention the molar ratio between the amino acids and sugars is from 0.5 to 2.0, like from 0.6 to 1.7, or from 0.7 to 1.4, or from 0.8 to 1.3 or from 0.9 to 1.1.

In some embodiments a melanoidin stock mixture is diluted to a desired concentration, and the obtained working melanoidin composition is applied to the plant to be protected or whose resistance should be improved. In some exemplary embodiments of the invention, the stock mixture is diluted with water. In some embodiments the melanoidin stock mixture comprises melanoidins of various structures and/or residual reactants and/or Maillard heating products. The amounts of brown melanoidin products can be easily calibrated and quantified toward standard melanoidin mixtures or other standard color materials, when needed. When the melanoidin stock mixture is diluted, for example 1000 fold in order to obtain the working melanoidin composition, any component of the mixture will be also diluted 1000 fold; throughout this text, such aqueous dilution will be denoted as “0.1% melanoidin working composition” or shortly as “0.1% melanoidin composition”, and the concentration will be related to as “melanoidin working concentration being 0.1%”. The concentration relates to the concentration of melanoidin (as well as any unreacted sugars and/or amino acids(s)).

According to a specific embodiment, the melanoidins are provided to plants in an aqueous mixture, for example in a solution, which may include additional agriculturally acceptable substances including, but not limited to, fertilizers, biostimulants, and pesticides (e.g., composts, manures, biochar, soilless media, growing papers, etc.). Said pesticides are selected from herbicide, acaricide, insecticide, antifungal, antibacterial, and antiviral.

According to various exemplary embodiments of the invention, various melanoidin types in solution or suspension or other formulation are applied by drench and/or by spray or sprinkle and/or formulated with fertilizers or pest control products or biostimulants or any other way to control a disease in plants and/or to protect a plant from other types of stress and support its healthy growth.

In some exemplary embodiments of a method according to the invention the melanoidin stock mixture is diluted to obtain between 0.01% and 0.5% working compositions, such as between 0.02% and 0.4%, or between 0.03% and 0.3% melanoidin working compositions.

In some embodiments melanoidins are applied to the plant to be treated at any frequency, for example, between once to three times per day. Alternatively, in some embodiments the plant may is treated only one time. In some embodiments melanoidins are provided to the plant on a regular basis, for example, as part of the irrigation or fertilization routine. According to a specific embodiment, the melanoidins are applied twice at two different days. According to another embodiment, the melanoidin treatment is supplemented by additional applications on various days. According to various exemplary embodiments of the invention, the melanoidins are applied to the plant or its part at any stage of its life cycle, including seed, germination, vegetative growth, flowering, and fruiting. A method is provided of preventing a disease in a plant belonging to the nightshades (Solanaceae), and to cucurbits (Curcubitaceae), or improving the resistance in said plants to said diseases, or reducing the symptoms of the diseases, or reducing the damages caused by the disease, wherein the disease is caused by virus, bacterium, oomycetes, or arthropod. In some exemplary embodiments of the invention, the melanoidin composition protects against at least two different diseases.

In some exemplary embodiments of the invention, a combined formulation comprising a melanoidin composition and at least one other known pesticide, wherein said at least two agents, melanoidin and other pesticide, are applied sequentially, simultaneously as two separate formulations, or simultaneously as one mixed formulation. According to these embodiments, use of melanoidin stock mixtures and/or melanoidin working compositions according to embodiments of the invention in agriculture provides previously unavailable means for fighting pests belonging to diverse groups. In the absence of treatment, these pests have the potential to cause heavy damage to nightshade and curcubit crops.

The melanoidin mixtures and composition are employed in several forms, including (i) as dry powder or granules to be admixed in any plant-suitable liquid with or without additives like surfactants, and applied as a liquid either by soil drench or foliar spray; (ii) as powder or granules applied directly to soil surface; (iii) as melanoidin form incorporated into slow release solids for soil application; (iv) as melanoidin form compounded together with other plant disease suppressive active ingredients; (v) as melanoidin form compounded together with fertilizers; (vi) adding to soil amendments, and more. According to various exemplary embodiments of the invention the melanoidins are supplied in solutions, suspensions, or otherwise formulated with inert diluents or carriers.

In some embodiments the melanoidin composition is applied by any of the known means of applying agents to a plant. For example, it is applied, formulated or unformulated, to any portion or part of the plant, including the foliage, stems, branches or roots, to the seed before it is planted or to other media in which plants are growing or are to be planted (such as the soil), directly or it may be sprayed on, dusted on, applied by dipping, applied through distribution or incorporation of a composition (such as a granular composition) in soil or an aqueous environment. The melanoidin composition according to the invention is preferably sprayed onto vegetation or applied by land or aerial irrigation systems. The melanoidin composition may be used in mixtures with fertilizers (for example nitrogen-, potassium- or phosphorus-containing fertilizers), which can be in the form of solution or solid.

In some embodiments, a melanoidin mixture prepared as described herein is employed mainly as a concentrated solid stock form, and before its use it is dispersed in water and diluted to the desired working concentration.

Various exemplary embodiments of the invention provide protection for a wide range of food crops against diverse pests without increasing the environmental burden, while keeping the costs very low, as the raw materials for manufacturing the melanoidin compositions are available and can be flexibly combined, and as they are entirely nontoxic. Importantly, the melanoidins are efficient at low concentrations. Both the reactants and the products are quite stable and easy for manipulation in agriculture. The active working composition can be easily obtained from concentrated stocks.

In some exemplary embodiments of the invention there is provided a composition comprising melanoidins and at least one other agrochemical. Said agrochemical is selected from pesticides, for example selected from known fungicides, herbicides, insecticides, or nematicides. In some embodiments, the additional fungicide may be a fungicidal sterol biosynthesis inhibitor, for example selected from the group consisting of prothioconazole, epoxiconazole, cyproconazole, myclobutanil, prochloraz, metconazole, difenoconazole, tebuconazole, tetraconazole, fenbuconazole, propiconazole, fluquinconazole, flusilazole, flutriafol, and fenpropimorph. In some embodiments, the sterol biosynthesis inhibitor is selected from the group consisting of prothioconazole, epoxiconazole, metconazole, difenoconazole, propiconazole, prochloraz, tetraconazole, tebuconazole, fenpropimorph, fenpropidin, ipconazole, triticonazole, spiroxamine, fenhexamid, and fenpyrazamine. In some embodiments, the sterol biosynthesis inhibitor is prothioconazole, in other embodiments the sterol biosynthesis inhibitor is epoxiconazole. In some embodiments, the sterol biosynthesis inhibitor is cyproconazole. In some embodiments, the sterol biosynthesis inhibitor is myclobutanil. In some embodiments, the sterol biosynthesis inhibitor is metconazole. In some embodiments, the sterol biosynthesis inhibitor is difenoconazole. In some embodiments, the sterol biosynthesis inhibitor is propiconazole. In some embodiments, the sterol biosynthesis inhibitor is prochloraz. In some embodiments, the sterol biosynthesis inhibitor is tetraconazole. In some embodiments, the sterol biosynthesis inhibitor is tebuconazole. In some embodiments, the sterol biosynthesis inhibitor is fluquinconazole. In some embodiments, the sterol biosynthesis inhibitor is flusilazole. In some embodiments, the sterol biosynthesis inhibitor is flutriafol. In some embodiments, the sterol biosynthesis inhibitor is fenpropimorph. In some embodiments, the sterol biosynthesis inhibitor is fenpropidin. In some embodiments, the sterol biosynthesis inhibitor is ipconazole. In some embodiments, the sterol biosynthesis inhibitor is triticonazole. In some embodiments, the sterol biosynthesis inhibitor is spiroxamin. In some embodiments, the sterol biosynthesis inhibitor is fenhexamid. In some embodiments, the sterol biosynthesis inhibitor is fenpyrazamine. In some embodiments, the sterol biosynthesis inhibitor is fenbuconazole.

In some embodiments the additional fungicide is a succinate dehydrogenase inhibitor. In some embodiments, the succinate dehydrogenase inhibitor is selected from the group consisting of benzovindiflupyr, penthiopyrad, isopyrazam, fluxapyroxad, boscalid, fluopyram, bixafen, and penflufen. In some embodiments, the succinate dehydrogenase inhibitor is benzovindiflupyr. In some embodiments, the succinate dehydrogenase inhibitor is penthiopyrad. In some embodiments, the succinate dehydrogenase inhibitor is isopyrazam. In some embodiments, the succinate dehydrogenase inhibitor is fluxapyroxad. In some embodiments, the succinate dehydrogenase inhibitor is boscalid. In some embodiments, the succinate dehydrogenase inhibitor is fluopyram. In some embodiments, the succinate dehydrogenase inhibitor is bixafen. In some embodiments, the succinate dehydrogenase inhibitor is penflufen.

In some embodiments, the additional fungicide is a strobilurin fungicide. In some embodiments, the strobilurin fungicide is selected from the group consisting of azoxystrobin, pyraclostrobin, picoxystrobin, fluoxastrobin, trifloxystrobin, kresoxim-methyl, dimoxystrobin, and orysastrobin. In some embodiments, the strobilurin fungicide is selected from the group consisting of azoxystrobin, pyraclostrobin, picoxystrobin, fluoxastrobin, and trifloxystrobin. In some embodiments, the strobilurin fungicide is azoxystrobin. In some embodiment the strobilurin fungicide is pyraclostrobin. In some embodiments, the strobilurin fungicide is picoxystrobin. In some embodiments, the strobilurin fungicide is fluoxastrobin. In some embodiments, the strobilurin fungicide is trifloxystrobin. In some embodiments, the strobilurin fungicide is kresoxim-methyl. In some embodiments, the strobilurin fungicide is dimoxystrobin. In some embodiments, the strobilurin fungicide is orysastrobin.

In some embodiments, said additional fungicide is a fungicidal multisite inhibitor. In some embodiments, the fungicidal multisite inhibitor is selected from a group consisting of mancozeb, chlorothalonil, folpet, captan, metiram, maneb, propineb, copper hydroxide, copper octanoate, copper oxychloride, copper sulfate, copper sulfate (tribasic), mancopper, oxine-copper, copper bis(3-phenylsalicylate), copper zinc chromate, cuprous oxide, cupric hydrazinium sulfate, and cuprobam. In some embodiments, the fungicidal multisite inhibitor is mancozeb. In some embodiments, the fungicidal multisite inhibitor is chlorothalonil. In some embodiments, the fungicidal multisite inhibitor is folpet. In some embodiments, the fungicidal multisite inhibitor is captan. In some embodiments, the fungicidal multisite inhibitor is metiram. In some embodiments, the fungicidal multisite inhibitor is maneb. In some embodiments, the fungicidal multisite inhibitor is propineb. In some embodiments, the fungicidal multisite inhibitor is copper hydroxide, copper octanoate, copper oxychloride, copper sulfate, copper sulfate (tribasic), mancopper, oxine-copper, copper bis(3-phenylsalicylate), copper zinc chromate, cuprous oxide, cupric hydrazinium sulfate, or cuprobam.

In some embodiments, the additional fungicide is selected from the group consisting of 2-(thiocyanatomethylthio)-benzothiazole, 2-phenylphenol, 8-hydroxyquinoline sulfate, ametoctradin, amisulbrom, antimycin, Ampelomyces quisqualis, azaconazole, azoxystrobin, Bacillus subtilis, Bacillus subtilis strain QST713, benalaxyl, benomyl, benthiavalicarb-isopropyl, benzylaminobenzene-sulfonate (BABS) salt, bicarbonates, biphenyl, bismerthiazol, bitertanol, bixafen, blasticidin-S, borax, Bordeaux mixture, boscalid, bromuconazole, bupirimate, calcium polysulfide, captafol, captan, carbendazim, carboxin, carpropamid, carvone, chlazafenone, chloroneb, chlorothalonil, chlozolinate, Coniothyrium minitans, copper hydroxide, copper octanoate, copper oxychloride, copper sulfate, copper sulfate (tribasic), cuprous oxide, cyazofamid, cyflufenamid, cymoxanil, cyproconazole, cyprodinil, dazomet, debacarb, diammonium ethylenebis-(dithiocarbamate), dichlofluanid, dichlorophen, diclocymet, diclomezine, dichloran, diethofencarb, difenoconazole, difenzoquat ion, diflumetorim, dimethomorph, dimoxystrobin, diniconazole, diniconazole-M, dinobuton, dinocap, diphenylamine, dithianon, dodemorph, dodemorph acetate, dodine, dodine free base, edifenphos, enestrobin, enestroburin, epoxiconazole, ethaboxam, ethoxyquin, etridiazole, famoxadone, fenamidone, fenarimol, fenbuconazole, fenfuram, fenhexamid, fenoxanil, fenpiclonil, fenpropidin, fenpropimorph, fenpyrazamine, fentin, fentin acetate, fentin hydroxide, ferbam, ferimzone, fluazinam, fludioxonil, flumorph, fluopicolide, fluopyram, fluoroimide, fluoxastrobin, fluquinconazole, flusilazole, flusulfamide, flutianil, flutolanil, flutriafol, fluxapyroxad, folpet, formaldehyde, fosetyl, fosetyl-aluminum, fuberidazole, furalaxyl, furametpyr, guazatine, guazatine acetates, GY-81, hexachlorobenzene, hexaconazole, hymexazol, imazalil, imazalil sulfate, imibenconazole, iminoctadine, iminoctadine triacetate, iminoctadine tris(albesilate), iodocarb, ipconazole, ipfenpyrazolone, iprobenfos, iprodione, iprovalicarb, isoprothiolane, isopyrazam, isotianil, kasugamycin, kasugamycin hydrochloride hydrate, kresoxium-methyl, laminarin, mancopper, mancozeb, mandipropamid, maneb, mefenoxam, mepanipyrim, mepronil, meptyl-dinocap, mercuric chloride, mercuric oxide, mercurous chloride, metalaxyl, metalaxyl-M, metam, metam-ammonium, metam-potassium, metam-sodium, metconazole, methasulfocarb, methyl iodide, methyl isothiocyanate, metiram, metominostrobin, metrafenone, mildiomycin, myclobutanil, nabam, nitrothal-isopropyl, nuarimol, octhilinone, ofurace, oleic acid (fatty acids), orysastrobin, oxadixyl, oxine-copper, oxpoconazole fumarate, oxycarboxin, pefurazoate, penconazole, pencycuron, penflufen, pentachlorophenol, pentachlorophenyl laurate, penthiopyrad, phenylmercury acetate, phosphonic acid, phthalide, picoxystrobin, polyoxin B, polyoxins, polyoxorim, potassium bicarbonate, potassium hydroxyquinoline sulfate, probenazole, prochloraz, procymidone, propamocarb, propamocarb hydrochloride, propiconazole, propineb, proquinazid, prothioconazole, pyraclostrobin, pyrametostrobin, pyraoxystrobin, pyrazophos, pyribencarb, pyributicarb, pyrifenox, pyrimethanil, pyriofenone, pyroquilon, quinoclamine, quinoxyfen, quintozene, Reynoutria sachalinensis extract, sedaxane, silthiofam, simeconazole, sodium 2-phenylphenoxide, sodium bicarbonate, sodium pentachlorophenoxide, spiroxamine, sulfur, SYP-Z048, tar oils, tebuconazole, tebufloquin, tecnazene, tetraconazole, thiabendazole, thifluzamide, thiophanate-methyl, thiram, tiadinil, tolclofos-methyl, tolylfluanid, triadimefon, triadimenol, triazoxide, tricyclazole, tridemorph, trifloxystrobin, triflumizole, triforine, triticonazole, validamycin, valifenalate, valiphenal, vinclozolin, zineb, ziram, zoxamide, Candida oleophila, Fusarium oxysporum, Gliocladium spp., Phlebiopsis gigantea, Streptomyces griseoviridis, Trichoderma spp., (RS)—N-(3,5-dichlorophenyl)-2-(methoxymethyl)-succinimide, 1,2-dichloropropane, 1,3-dichloro-1,1,3,3-tetrafluoroacetone hydrate, 1-chloro-2,4-dinitronaphthalene, 1-chloro-2-nitropropane, 2-(2-heptadecyl-2-imidazolin-1-yl)ethanol, 2,3-dihydro-5-phenyl-1,4-dithi-ine 1,1,4,4-tetraoxide, 2-methoxyethylmercury acetate, 2-methoxyethylmercury chloride, 2-methoxyethylmercury silicate, 3-(4-chlorophenyl)-5-methylrhodanine, 4-(2-nitroprop-1-enyl)phenyl thiocyanateme, ampropylfos, anilazine, azithiram, barium polysulfide, Bayer 32394, benodanil, benquinox, bentaluron, benzamacril, benzamacril-isobutyl, benzamorf, binapacryl, bis(methylmercury) sulfate, bis(tributyltin) oxide, buthiobate, cadmium calcium copper zinc chromate sulfate, carbamorph, CECA, chlobenthiazone, chloraniformethan, chlorfenazole, chlorquinox, climbazole, copper bis(3-phenylsalicylate), copper zinc chromate, cufraneb, cupric hydrazinium sulfate, cuprobam, cyclafuramid, cypendazole, cyprofuram, decafentin, dichlone, dichlozoline, diclobutrazol, dimethirimol, dinocton, dinosulfon, dinoterbon, dipyrithione, ditalimfos, dodicin, drazoxolon, EBP, ESBP, etaconazole, etem, ethirim, fenaminosulf, fenapanil, fenitropan, fluotrimazole, furcarbanil, furconazole, furconazole-cis, furmecyclox, furophanate, glyodine, griseofulvin, halacrinate, Hercules 3944, hexylthiofos, ICIA0858, isopamphos, isovaledione, mebenil, mecarbinzid, metazoxolon, methfuroxam, methylmercury dicyandiamide, metsulfovax, milneb, mucochloric anhydride, myclozolin, N-3,5-dichlorophenyl-succinimide, N-3-nitrophenylitaconimide, natamycin, N-ethylmercurio-4-toluenesulfonanilide, nickel bis(dimethyldithiocarbamate), OCH, phenylmercury dimethyldithiocarbamate, phenylmercury nitrate, phosdiphen, prothiocarb, prothiocarb hydrochloride, pyracarbolid, pyridinitril, pyroxychlor, pyroxyfur, quinacetol, quinacetol sulfate, quinazamid, quinconazole, rabenzazole, salicylanilide, SSF-109, sultropen, tecoram, thiadifluor, thicyofen, thiochlorfenphim, thiophanate, thioquinox, tioxymid, triamiphos, triarimol, triazbutil, trichlamide, urbacid, zarilamid, and any combinations thereof.

In some embodiments, the additional fungicide is a botryticide selected from Pyrimethanil, Cyprodinil, Mepanipyrim, Iprodione, procymidone, fludioxonil, thiophanate-methyl, benomyl, carbendazim, diethofencarb, chlorothalonil, dichlofluanid, folpet, thiram, fluazinam, azoxystrobin, pyraclostrobin, Boscalid, Fluopyram, Prochloraz, tebuconazole, Fenhexamide, Fenpyrazamine

It is expected that during the life of this patent many new techniques for applying powders and/or liquids to crops will be developed and the scope of the invention is intended to include all such new technologies a priori.

As used herein the term “about” refers to ±10%.

As used herein the term “substantially” means within the normal limits of detection.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

Specifically, a variety of numerical indicators have been utilized. It should be understood that these numerical indicators could vary even further based upon a variety of engineering principles, materials, intended use and designs incorporated into the various embodiments of the invention. Additionally, components and/or actions ascribed to exemplary embodiments of the invention and depicted as a single unit may be divided into subunits. Conversely, components and/or actions ascribed to exemplary embodiments of the invention and depicted as sub-units/individual actions may be combined into a single unit/action with the described/depicted function.

Alternatively, or additionally, features used to describe a method can be used to characterize a composition and features used to describe a composition can be used to characterize a method.

It should be further understood that the individual features described hereinabove can be combined in all possible combinations and sub-combinations to produce additional embodiments of the invention. The examples given above are exemplary in nature and do not limit the scope of the invention which is defined solely by the following claims.

Each recitation of an embodiment of the invention that includes a specific feature, part, component, module or process is an explicit statement that additional embodiments of the invention not including the recited feature, part, component, module or process exist.

Alternatively or additionally, various exemplary embodiments of the invention exclude any specific feature, part, component, module, process or element which is not specifically disclosed herein.

Specifically, the invention has been described in the context of nightshades and cucurbits but might also be used in the context of other crops.

All publications, references, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

Priority application IL 267627 is fully incorporated herein by reference.

The terms “include”, and “have” and their conjugates as used herein mean “including but not necessarily limited to”.

Additional objects, advantages, and novel features of various embodiments of the invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

### EXAMPLES

Reference is now made to the following examples, which together with the above descriptions, illustrate the invention in a non limiting fashion.

### Example 1

Following the Melanoidins Formation

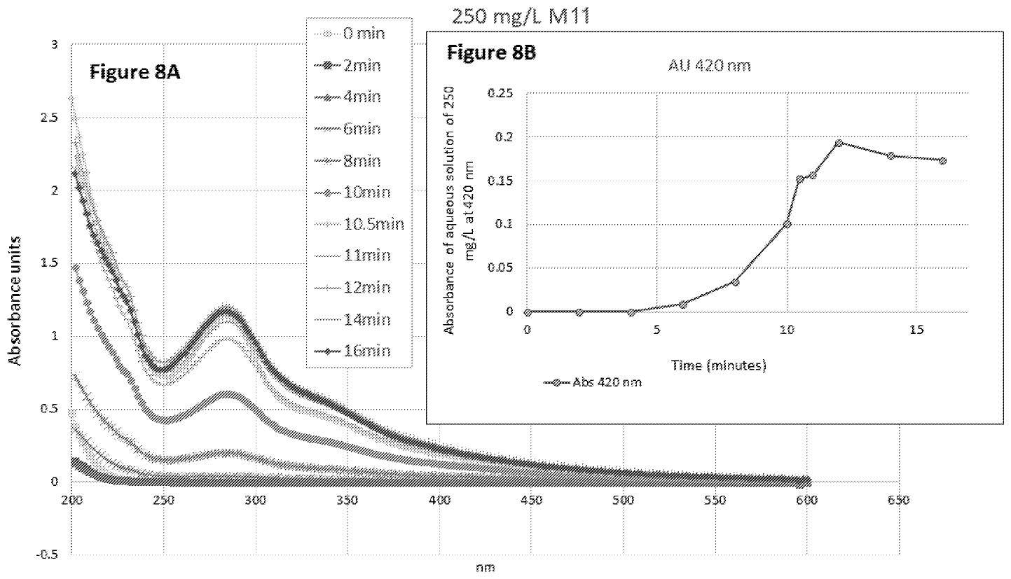

The Maillard reaction is a class of non-enzymatic browning reactions that involves the interaction of reducing sugars and free amino acids or a free amino group of an amino acid that is a part of the protein chain. It is known that the reaction is affected by the temperature and composition of the reacting mixture, while both UV-absorbing and colorless intermediates are formed at the initial stages and the brown melanoidins, oligomeric or polymeric, at final ones. Melanoidins may be quantified by determining absorbance at 420 nm as a measure of ‘browning’ or by determining absorbance at other wavelength as a follow-up of the reaction. FIG. 8A shows the UV-Vis absorbance spectra of twelve samples obtained by i) heating a solid mixture of glucose and glutamic acid (called M11) in a molar ratio of 1:1, ground and heated in an oven at 170° C. for different time intervals: 0, 2, 4, 6, 8, 10, 10.5, 11, 12, 14, and 16 minutes; and ii) dispersing the product of the heating water at a concentration of 250 mg/L, and measuring the absorbance of the dissolved part. Portions heated for 11, 12, 14 and 16 minutes were nearly blackened in color and were less soluble in water (less than 250 mg/L), in contrast with much higher solubility of melanoidins. FIG. 8B (inset) shows spectra of the samples. A maximum in the spectra can be seen at about 284 nm. The results show that the soluble and UV-absorbing products of heating the sugar/amino acid mixture reach a maximal absorbance value at a certain time, for all wavelength values. FIG. 8B shows that absorbance at 420 nm reaches a plateau after about 10.5 minutes under the employed specific conditions. It was found that the solubility of the heating products decreased after reaching this plateau, indicating the formation of less desired products, under the specific employed conditions. In one aspect of the invention, a powder of glucose and glutamic acid is heated at a temperature of between 120 and 180° C., such as 150 or 170° C., for a time necessary to form melanoidins without excessive formation of less desired products.

It is expected that reaction kinetics will vary with reaction scale and/or specific heating equipment. Regardless of the scale or equipment, conditions which provide the desired melanoidin yield can be determined empirically using FIGS. 8A and 8B and the descriptive text above as a guide.

Preparing and Using Melanoidin Compositions

A) In some experiments, a solid commercial protein hydrolysate was ground together with one or more solid reducing sugars at a 1:1 weight ratio of hydrolysate to sugar, providing approximately an equimolar mixture, while obtaining a fine homogeneous powder. The mixture was heated for 8.5 minutes at 150° C. in a pre-heated oven, and then transferred to a desiccator with silica gel for cooling to room temperature. During the heating, the amino acids in the hydrolysate reacted with the sugars to form brown-colored melanoidins via the Maillard reaction. The cooled melanoidin mixture was gently ground and transferred to hermetically sealed vials for storage.

B) In other experiments, commercial L-glutamic acid and D-glucose were ground together at an equimolar ratio to obtain a fine homogeneous powder. The mixture was heated for 8.5 minutes at 150° C. in a pre-heated oven, and then transferred to a desiccator with silica gel for cooling to room temperature. During the heating, the amino acid reacted with the sugar to form brown-colored melanoidins product via the Maillard reaction. The cooled melanoidin mixture was gently ground and transferred to hermetically sealed vials for storage as a stock melanoidin mixture.

The melanoidins obtained in the method according to embodiments of the invention had a high solubility in water, such as greater than 10 g/L or even greater than 100 g/L at 25° C. The high solubility ensures that the active components remain in the solution even if some components of working compositions exist as solids, for example in suspension, particularly when including additional agents in some embodiments of the working compositions. The melanoidin stock mixtures were diluted with water to obtain melanoidin working concentrations of between 0.02% and 0.2%. The melanoidin working compositions of 0.03% and 0.1% were employed in many tests. The treatment with melanoidin composition consisted of either spray or drench at a volume of 5 mL/plant. Treatments were done twice prior to pest or pathogen infection: 3 days and 4 hours prior to the inoculation.

Treatments in experiments were replicated 5-10 times. Replicates of each treatment were arranged randomly in the greenhouse or growing chamber. Disease severity data in percentages were arcsin-transformed before further analysis. Disease severity data were analyzed using ANOVA and Fisher's protected LSD test. Standard errors (SE) of the means were calculated and disease levels were statistically separated (P0.05) following a one-way analysis of variance. Control plants were treated by water and kept under the same conditions as mentioned above.

### Example 2

Plants and Diseases Evaluation

Generally, tomato plants were grown from seeds in a nursery and transplanted into 1 liter pots at 40 to 50 days after seeding. Plants were fertilized with NPK fertilizer (irrigation water aimed at total N, P and K concentrations of 120, 30 and 150 mg/L, respectively). Plants were usually maintained at 20 to 30° C. with natural light, and relative humidity of 50-90% in a pest- and disease-free greenhouse during the growth period and then transferred to an area where diseases were allowed to develop following pathogen infection on intact or detached leaves as described below. Disease severity was evaluated on each plant according to a % coverage key, whereby 0% is defined as all leaves with no disease symptoms and 100% is defined as all leaves fully covered by disease symptoms. This same coverage key is used throughout.

### Example 3

Virus Disease

Tomato brown rugose fruit virus (TBRFV) was maintained on tomato plants. For the inoculation, the infected plant parts were blended to small pieced in tap water using a blender, and the crushed plant material was sprayed with carborundum dust on the treated tomato plants. The plants were kept in a greenhouse with 18-28° C. Disease severity was evaluated on each plant according to a % coverage key, whereby 0% is defined as all leaves with no disease symptoms and 100% is defined as all leaves fully covered by disease symptoms.

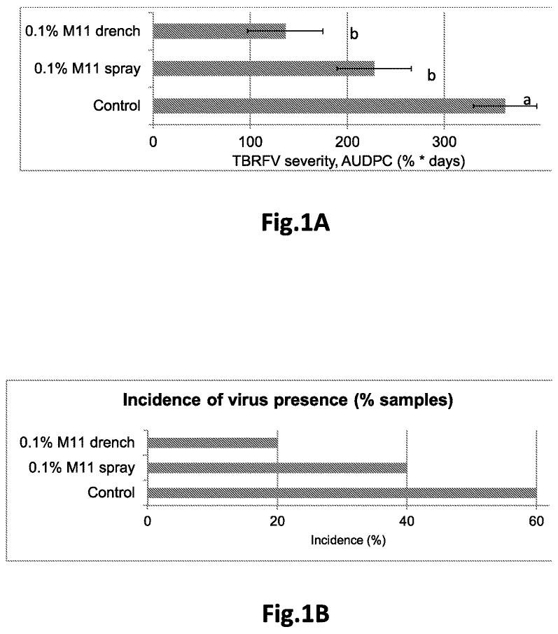

TBRFV causes symptoms that include a mosaic pattern on leaves accompanied occasionally with narrowing of leaves and with yellow spotted, wrinkled fruit. TBRFV is a virus that belongs to the Tobamovirus genus. It was found that spraying the melanoidin composition according to the invention on tomato plants or drenching the composition to the root zone of tomato plants significantly suppressed the severity of the virus infection as manifested on the plant canopy (FIG. 1).

### Example 4

Virus Disease

Tomato yellow leaf curl virus (TYLCV) was maintained on tomato plants. Spread of the disease onto treated tomato plants was achieved by the migration of the insect vector, Bemisia tabaci, from the infected plants. The plants were kept in a greenhouse at 18-28°. Disease severity was evaluated on each plant according to a % coverage key, whereby 0% is defined as all leaves with no disease symptoms and 100% is defined as all leaves fully covered by disease symptoms.

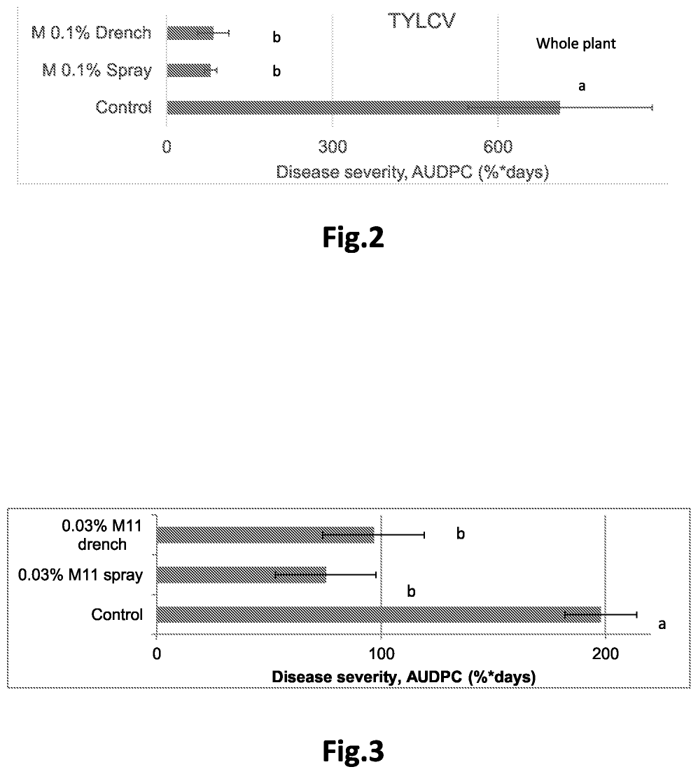

TYLCV is a DNA virus from the genus Begomovirus, transmitted by an insect vector—Silverleaf whitefly (Bemisia tabaci). The infected plants include tomato, eggplant, potato, tobacco, and pepper. Virus infection causes stunting, reduction of leaf size, upward cupping/curling of leaves, chlorosis on leaves and flowers, and reduction of fruit production. It was found that spraying the melanoidin composition according to the invention on tomato plants or drenching the composition to the root zone of tomato plants significantly suppressed the severity of the virus infection on the tomato plants as manifested on the plant canopy (FIG. 2).

### Example 5

Bacterial Disease

Pseudomonas syringae pv. tomato (Okabe), causing tomato bacterial speck, was isolated from a diseased plant. The bacterium was grown and maintained on nutrient agar. Cell suspension (10⁵/ml) in tap water was sprayed on the plants after treatments with melanoidins. The tomato plants were whole plants kept in a humidity chamber at 20±1° C., 97±3% RH, and 1020 lux light intensity. Disease severity was evaluated on each plant using a pictorial key; 0=no infection (all leaves are symptomless) and 100=all leaves are fully covered by bacterial speck lesions.

The bacterium causes small spots (specks) on the leaves of the tomato plant, which are brown in the center and surrounded by a yellow ring. In severe symptoms the spots overlap and look larger and irregular and spread to the fruit. It was found that spraying the melanoidin composition according to the invention onto tomato plants or drenching the composition to the root zone of tomato plants significantly suppressed the severity of P. syringae pv. tomato speck disease as manifested on the leaves (FIG. 3).

### Example 6

Insect Disease

The moth Tuta absoluta Meyrick lay eggs on the leaves of tomato plants. Those eggs hatch to larva, which eat the leaves in a process known as leaf mining. In this experiment, the moth infestation was natural. The plants were kept in a greenhouse with 18-28° C. Mining symptoms severity were evaluated on each plant; 0=no infection (all leaves are symptomless) and 100=leaves were fully covered by symptoms.

The insect larva feeds on tomato plants, especially on leaves, producing large galleries in the leaflets and feeding on young fruits and apical buds. Tomato is the main host plant, but T. absoluta also attacks other crop plants including potato, eggplant, pepper, tobacco, and other Solanaceous plants. The insect is capable of causing total yield loss.

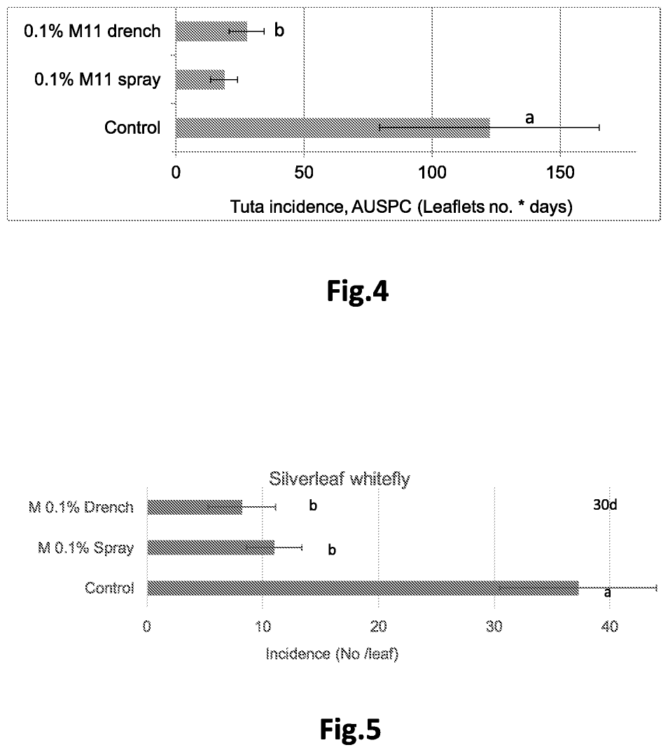

It was found that the severity of the leaf miner damages on the tomato plants treated by spraying or drenching with the melanoidin composition according to the invention was significantly suppressed by each of the treatments (FIG. 4).

### Example 7

Insect Pest

Silverleaf whitefly (Bemisia tabaci Gennadius), also called sweet potato whitefly, occurred naturally on the treated tomato plants. The plants were kept in a greenhouse with 18-28° C. Incidence of insect individuals number on the 10th leaf from the bottom of the tomato plants were counted.

The insect causes damage to many plant crops by feeding on them and by transmission of virus plant pathogens. It sucks phloem liquid from the leaves, causes whitish dots and secretes honeydew that promotes the development of sooty molds on plant canopies. Many crop plants may be affected. It was found that the incidence of the Silverleaf whitefly on tomato leaves of plants treated by spraying or drenching with the melanoidin composition according to the invention was significantly suppressed (FIG. 5).

### Example 8

Arachnid Pest

The red spider mite, Tetranychus urticae Koch, also known as two-spotted spider mite, occurred naturally on the treated tomato plants. The plants were kept in a greenhouse between 18-28° C. Typical scratching symptoms severity were evaluated on each plant; 0=no infection (all leaves are symptomless) and 100=leaves were fully affected by symptoms.

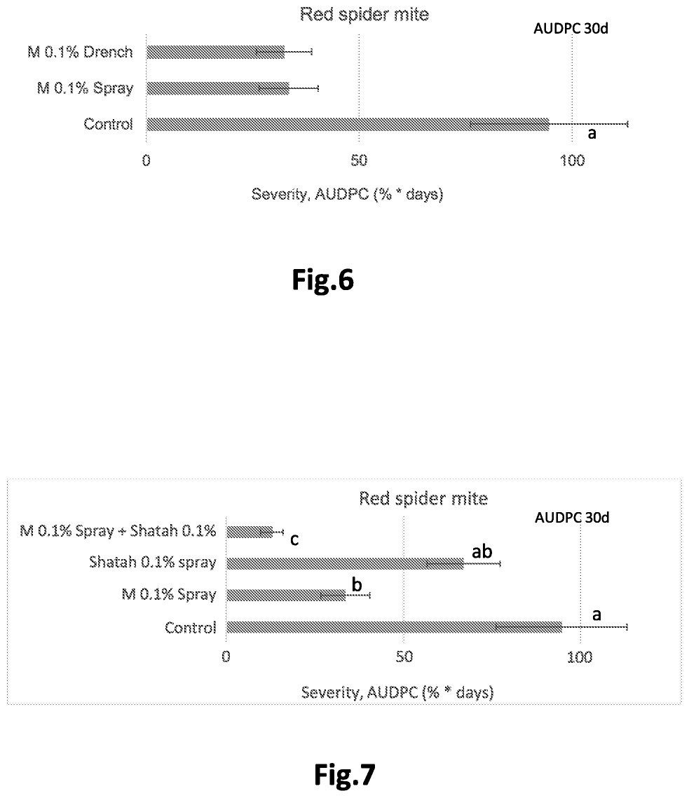

Tetranychus urticae Koch is a plant feeding mite of the family Tetranychidae, which can feed on tomato, pepper, and potato. It sucks the cell contents in leaves causing whitish spots on the leaf surface. Eventually it reduces the photosynthetic ability of the plants, eventually causes leaf mortality and major yield losses. It was found that severity of damage caused by the mite on tomato leaves treated by spraying or drenching with the melanoidin composition according to the invention was significantly suppressed by each of the treatments (FIG. 6).

### Example 9

Combined Treatment

A combination of melanoidins with adjuvant Shatah (containing 92% alkyl phenol ethylene oxide condensate (Adama Ltd., Israel) in suppressing red spider mite Tetranychus urticae on tomato plants was found very efficient.

It was found that the melanoidin composition of 0.1% sprayed according to the invention on tomato canopy reduced the damages caused by the red spider mite (FIG. 7, see b versus control a). A spray with 0.1% Shatah showed lower effect than the melanoidin composition (see c versus b). However, the combination of both agents, melanoidin treatment with the adjuvant alkyl phenol ethylene oxide spray, resulted in a significantly better result than each of the treatments alone (FIG. 7, compare d versus b or c). The melanoidin solution and the adjuvant solution were applied at 3 days before and 4 hours before inoculation and incubation of the plants at a greenhouse with 18-28° C. Severity of red spider mite symptoms was evaluated on a scale of 0-100% severity of symptoms coverage during 30 days after treatment and it is expressed as the area under disease progress curve (AUDPC). Bars=Standard Errors; Columns followed by a different letter are significantly different (P≤0.05).

In other tests, combined treatments comprising melanoidins and synthetic pesticides were examined and found as more efficient than the treatments with single agents. The fact is that combining more pesticide types may improve the pest suppression, reduce the amounts of applied toxins, and prevent the development of resistance in the pests towards the pesticides. Among the pesticides combined with melanoidins there were, for example, pyrimethanil, cyprodinil, fludioxonil.

### Example 10

Oomycetes Test

Oomycetes are a class of microorganisms containing cellulose in their cell walls, they are diploid in their vegetative state, and they contain coenocytic hyphae (lacking crosswalls); they reproduce asexually with motile biflagellate zoospores that require water to move; they reproduce sexually with structures called antheridia, oogonia, and oospores. Effect of melanoidins on the oomycete disease cucurbits downy mildew (Pseudoperonospora cubensis, Berk. & Curtis, Rostovzev) in cucumber plants was examined. P. cubensis is a species of Oomycete that causes downy mildew on cucurbits such as melon, cantaloupe, cucumber, pumpkin, squash, and watermelon. It is an important pathogen of these crops that is promoted by high humidity. The pathogen causes angular chlorotic lesions on the leaves that are bound by leaf veins. The underside of the leaf bears gray-brown to purplish-black hyphal growth with conidiation. Leaves turn necrotic and the entire canopy may die. Oomycete P. cubensis (Berk. & Curtis) Rostovzev was isolated from infected cucumber plants in water. Cells of the oomycete were suspended in tap water (103/m1) and sprayed onto treated cucumber plants. The tomato plants were whole plants kept in a humidity chamber at 20±1° C. of 97±3% RH, and 1020 lux light intensity. During the first and seventh days of incubation the plants were kept in the dark for 12 hours in order to allow infection and sporulation, respectively. Disease severity was evaluated on each plant using a pictorial key; 0=no infection (all leaves are symptomless) and 100=all leaves are fully covered by downy mildew lesions.

The melanoidins solution was applied by spraying on the cucumber plants at 3 days before and 4 hours before inoculation and incubation of the plants at high humidity conditions and one week later. Conidia suspension of the pathogen was sprayed on the plants immediately before incubation. Disease severity was evaluated during 23 days after treatment and it is expressed as the area under disease progress curve (AUDPC). It was found that 0.1% aqueous melanoidin spray on cucumber plants significantly suppressed the severity of downy mildew on cucumber leaves, from severity 92, down to 33.

### Example 11

Oomycetes Test

Effect of melanoidins on the oomycete Pythium aphanidermatum (Edson) Fitz. damping off in cucumber seedlings was examined. P. aphanidermatum is a soil borne plant pathogen, a genus in the class Oomycetes. It has a wide host range, being of an economic impact on the cultivation of many plants and crops. It is a major cause of root rot, pre- and post-emergence damping off. P. aphanidermatum inoculum grown in the laboratory under sterile conditions was mixed with soil. Seedlings of cucumber were planted (4 seedlings per pot, 5 pots, 1.5 L each) in the infested soil 7 days after seeding in non-infested soil. The plants were kept at 24±1.5 C. Seedlings were treated by drench and by spray of 0.1% melanoidines solution. First treatment was applied at seedlings age of 3 days, 2nd treatment on 7 days-old seedlings and the last treatment was applied on the transplanted seedlings 4 days later (day 11). The number of plants affected by damping off was counted and the percentage of damping off was calculated. Seedlings were transplanted at the age of 7 days after seeding into P. aphanidermatum infested soil. The melanoidins solution was applied by spraying on the cucumber seedlings and by drenching to the root zone at the age of 3 days, 7 days and 11 days. Damping off incidence was counted during 8 days after transplanting and it is expressed as the area under mortality progress curve through 8 days. It was found that sprayed and drenched melanoidins solution on cucumber plants significantly suppressed the incidence of P. aphanidermatum damping off in cucumber seedlings, reducing the diseases incidence (expressed in %*days) from 125 in control down to 70 for spray and 20 for drench.

### Example 12

Oomycetes Test

Phytophthora infestans (Mont.) de Bary is an oomycete that causes a serious tomato disease named late blight, being a major disease of potato and other nightshade crops that is favored by moisty and cool environment. Tomato plants were grown in 10 L growing medium buckets in a net house naturally infected plants grown in a net house with 23±7° C. were treated by drench or by spraying of melanoidines on a weekly basis starting from 30 days after planting and for 7 times more. The disease occurred naturally from 16 days after first treatment. Disease severity was evaluated on a scale of 0-100% severity of symptoms coverage during 64 days after first treatment.

Effects of 0.03 or 0.1% melanoidins solution in spray or drench on the severity of late blight caused by the oomycete on tomato plants were examined. The melanoidins solution was applied by drench and by spraying on the tomato plants on a weekly basis starting from 30 days after planting and for 7 times more. Disease occurred naturally from 16 days after first treatment. Disease severity was evaluated on a scale of 0-100% severity of symptoms coverage during 64 days after first treatment and it is expressed as the area under disease progress curve.

It was found that sprayed and drenched melanoidins solution on tomato plants significantly suppressed the severity of P. infestans late blight on tomato canopy. The disease severity (expressed in %*days units) was reduced from 740 for the control, down to 510, 420, 300, and 180, for 0.1% spray, 0.03% drench, 0.1% drench, and 0.03% spray, respectively.

While the invention has been described using some specific examples, many modifications and variations are possible. The invention is therefore not limited in any way, other than by the scope of the appended claims.

1. A composition comprising a melanoidin.

2. A composition according to claim 1 comprising at least one additional component selected from the group consisting of insecticides, ascaricides and antiviral compounds.

3. A composition according to claim 1 or claim 2, wherein said melanoidin is obtained by reacting an amino acid source and a reducing sugar source in solid phase or in solution or in suspension at a temperature of from 110° C. to 180° C.

4. A composition according to claim 3, wherein said reaction proceeds at least 20% to completion as evaluated spectrophotometrically.

5. A composition according to any one of claims 3 to 4, wherein said amino acid source comprises one or more amino acids, one or more peptides with a free amino group, or a protein hydrolysate.

6. A composition according to any one of claims 3 to 5, wherein said reducing sugar source comprises one or more monosaccharides, one or more disaccharides, one or more oligosaccharides, or mixtures thereof.

7. A composition according to any one of claims 1 to 6, provided as a powder.

8. A composition according to any one of claims 1 to 7, provided as a solution or a suspension in which said melanoidin constitutes from 0.002 to 2 wt %, to be used as a working composition.

9. A composition according to claim 8, wherein said melanoidin constitutes from 0.01 to 1 wt %.

10. A composition according to any one of claims 2 to 9, wherein said additional component Is selected from pyrimidine-based agents, phenylpyrrole-based agents, alkyl phenol ethylene oxide condensate, pyrimethanil, cyprodinil, or fludioxonil.

11. A composition according to any one of claims 1 to 10, labeled for use in treatment of a nightshade or curcubit crop for a non-fungal pest.

12. A composition according to claim 11, wherein said nightshade crop comprises tomato.

13. A composition according to claim 11, wherein said curcubit crop comprises cucumber.

14. A composition according to any one of claims 1 to 10, for use in the treatment of a nightshade crop for a non-fungal pest.

15. Use according to claim 14, wherein said non-fungal pest includes at least one member of the group consisting of viruses, bacteria, oomycetes, and arthropods.

16. Use according to claim 15, wherein said pests comprise at least two different pests selected from said group.

17. A crop treatment method comprising:
applying a formulation including a brown melanoidin product to crops infected by, or at risk for infection by, at least one member of the group consisting of viruses, bacteria, oomycetes, and arthropods.

18. A crop treatment method according to claim 17, wherein said brown melanoidin product is the result of reaction of an amino acid with a reducing sugar at a molar ratio of between 0.5 and 2.0 at a temperature of from 120° C. to 180° C., for a time period sufficient to achieve melanoidinization between 0.2 and 1 when measured spectrophotometrically.

19. The method of any one of claims 17 to 18, wherein said formulation is applied as an aqueous solution with a brown melanoidin concentration of from 0.002 to 2 wt %.

20. The method of claim 19, wherein the brown melanoidin concentration is from 0.01 to 1 wt %.

21. The method of any one of claims 17 to 18, wherein said applying is via an application route selected from the group consisting of spraying, sprinkling, drenching, irrigation, and fertigation.

22. A melanoidin composition for use in the treatment of a nightshade or curcubit crop to protect against a non-fungal pest.

23. Use according to claim 22, wherein said non-fungal pest includes at least one member of the group consisting of viruses, bacteria, oomycetes, and arthropods.

24. Use according to claim 22 or 23, wherein said nightshade crop is tomato.

25. Use according to any one of claims 22 to 24, wherein said non-fungal pest is Tomato brown rugose fruit virus (TBRFV).

26. Use according to any one of claims 22 to 24, wherein said non-fungal pest is Tomato yellow leaf curl virus (TYLCV).

27. Use according to any one of claims 22 to 24, wherein said non-fungal pest is Pseudomonas syringae pv. Tomato.

28. Use according to any one of claims 22 to 24, wherein said non-fungal pest is Tuta absoluta.

29. Use according to any one of claims 22 to 24, wherein said non-fungal pest is Bemisia tabaci Gennadius.

30. Use according to any one of claims 22 to 24, wherein said non-fungal pest is Tetranychus urticae.

31. Use according to any one of claims 22 to 24, wherein said non-fungal pest is Phytophthora infestans.

32. Use according to claim 22 or 23, wherein said curcubit crop is cucumber.

33. Use according to any one of claim 22, 23 or 32, wherein said non-fungal pest is Pseudoperonospora cubensis.

34. Use according to any one of claim 22, 23 or 32, wherein said non-fungal pest is Pythium aphanidermatum.
